(19) United States
(12) Patent Application Publication
(10) Pub. No.: US 2020/0046561 A1
(43) Pub. Date: Feb. 13, 2020

(54) Safety Eyewear for Reclining and Supine Positions

(71) Applicant: Thomas A.J. Basora, (US)

(72) Inventors: Thomas A. J. Basora, Sparks, NV (US); Julian Ballatore-South, Reno, NV (US)

(73) Assignee: Thomas A. J. Basora

(21) Appl. No.: 16/532,705

(22) Filed: Aug. 6, 2019

Related U.S. Application Data
(60) Provisional application No. 62/833,914, filed on Apr. 15, 2019.
(60) Provisional application No. 62/715,786, filed on Aug. 7, 2018.

(51) Int. Cl. A61F 9/02; G02C 5/12; G02C 3/00

(52) U.S. Cl. CPC A61F 9/026; A61F 9/022; G02C 3/003; G02C 5/126

(57) ABSTRACT

Safety eyewear that is particularly suitable for patients undergoing medical or dental procedures in reclining or supine positions is disclosed. The safety eyewear has a frame configured to rest over a face. The frame includes a pair of lenses separated from one another by a bridge. The lenses may be configured to block certain harmful wavelengths of light used in dental curing lights, patient overhead lights, and lasers. One of both of the frame and the pair of lenses may curve in a posterior direction along inferior edges so as to terminate closely proximate to the face. Left and right temple arms extend generally straight in the posterior direction from respective left and right sides of the frame. A nasion pad is coupled to the bridge of the frame and extends in the posterior direction from the bridge to make contact with the patient's nasion.

### CROSS-REFERENCE TO RELATED APPLICATIONS

This application claims priority to U.S. Provisional Patent Application No. 62/715,786, filed Aug. 7, 2018, and to U.S. Provisional Patent Application No. 62/833,904, filed Apr. 15, 2019. The contents of both of those applications are incorporated by reference in their entireties.

### TECHNICAL FIELD

The invention relates to safety eyewear, and in particular, to safety eyewear for dental and medical patients who are in a reclining or fully supine position.

### BACKGROUND

During dental procedures, patients are exposed to many potential hazards, including dropped instruments, dental materials, flying debris from high-speed rotary tools, high-intensity composite resin curing lights, and lasers, to name a few. For that reason, safety regulations increasingly require dentists to place safety glasses over the eyes of patients. Safety glasses may have additional benefits as well—for example, darkened or filtered safety glasses may reduce patient anxiety and make it easier for the clinician to work.

For over 20 years, clinicians and others have recognized the safety hazards to which dental patients may be exposed. For example, U.S. Pat. No. 5,413,119 to George Guerrant describes protective eyewear for dental patients and was issued in 1995. Despite the recognition of the issues, designers have struggled to produce safety eyewear that works well for patients in a reclining or fully supine position, as dental patients typically are during treatment.

One problem is that most safety eyewear—and most eyewear in general—is designed for use in an upright position. In that position, the eyewear rests on the bridge of the nose, and temple arms extend posteriorly to hook over the ears. This keeps the eyewear stable on the face, with the force of gravity causing the frame of the eyewear to bear down on the ears and on the bridge of the nose.

When the patient is in a reclining or supine position, the situation is entirely different. The eyewear may shift on the face, and rearward pressure on the ends of the temples may force the eyewear away from the face, reducing or eliminating its effectiveness as safety eyewear.

The eyewear disclosed in the Guerrant patent uses a strap that is secured around the head to address some of these issues, but that strap may be uncomfortable, and while a strap may place more pressure on the eyewear and on the head, it does not address the fundamental issues of eyewear stability in the reclining or supine position—i.e., the eyewear is still not well-fitted to the face. In practice, some clinicians resort to stopgap fixes like placing rolls of gauze under the frame of the eyewear, particularly at the bridge of the nose. That approach can be distracting, uncomfortable, and frustrating for both the patient and the clinician, especially if the clinician is forced to reposition the gauze or the safety glasses during the procedure. Fundamentally, poorly-fitting safety eyewear may not provide adequate protection for the patient's eyes.

### SUMMARY OF THE INVENTION

Aspects of the invention relate to safety eyewear for patients in reclining and supine positions. Safety eyewear according to these aspects of the invention may be particularly suitable for use during dental and medical procedures in which the patient is reclining or in a supine position.

Safety eyewear according to this aspect of the invention typically comprises a frame in which a pair of lenses are provided. The lenses may be adapted to filter specific wavelengths of light or may have other safety features to protect against high-intensity composite resin curing lamps, lasers, and other such optical hazards. A temple arm is provided on each side of the frame. The temple arms extend generally straight and may terminate in relatively large, round pads without bending over the ears. The safety eyewear may have curvature laterally as well as in the superior-inferior direction, for example, wrapping around the face laterally, and terminating close to or resting against the face above the brow and/or near the cheekbone. The close proximity to the face may prevent ingress of debris between the safety eyewear and the face while potentially also accommodating long eyelashes and providing for greater patient comfort. In some embodiments, the safety eyewear may rest against the face along its inferior edges, close to the mouth, and may have more distance between the superior edge or edges and the face.

The safety eyewear may also provide a better fit around the nasion, the anatomical feature just above the bridge of the nose. More specifically, the safety eyewear according to this aspect of the invention includes a nasion pad that is configured to make contact with the face at the nasion and to support the safety eyewear against it. The nasion pad may be removable and replaceable or disposable. For example, it may take the form of a clip that inserts over the bridge of the safety eyewear and extends downwardly.

Another aspect of the invention also relates to safety eyewear. Safety eyewear according to this embodiment of the invention has a frame, lenses, and temple arms as described above. However, the frame and lenses are not as deeply contoured and are not themselves intended to rest against the face. Therefore, in order to prevent ingress of debris between the safety eyewear and the face, superior and inferior frame supports are installed along the superior and inferior edges of the frame. The frame supports may, e.g., be made of a soft rubber, intended to rest against the face comfortably and to make a seal between the face and the safety eyewear. A central portion of the superior frame support forms a nasion pad, intended to rest against the nasion.

Yet other aspects of the invention relate to nasion pads and frame supports that are provided in kit form, to be attached to more traditional forms of safety eyewear in order to add stability in reclining and supine positions.

Other aspects, features, and advantages of the invention will be set forth in the description that follows.

### BRIEF DESCRIPTION OF THE DRAWING FIGURES

The invention will be described with respect to the following drawing figures, in which like numerals represent like features, and in which:

FIG. 1 is a perspective view of a patient in a reclining position wearing safety eyewear according to one embodiment of the present invention;

FIG. 2 is a perspective view of the safety eyewear of FIG. 1 in isolation;

FIG. 3 is a cross-sectional view of the eyewear of FIG. 1, shown on a patient;

FIG. 4 is an exploded perspective view of the safety eyewear of FIG. 1, illustrating the installation of alternative nasion pads; and

FIG. 5 is an exploded perspective view of safety eyewear according to another embodiment of the invention.

### DETAILED DESCRIPTION

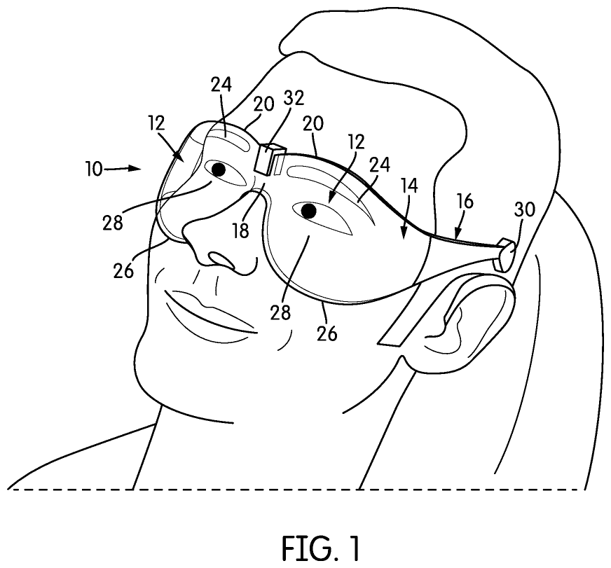

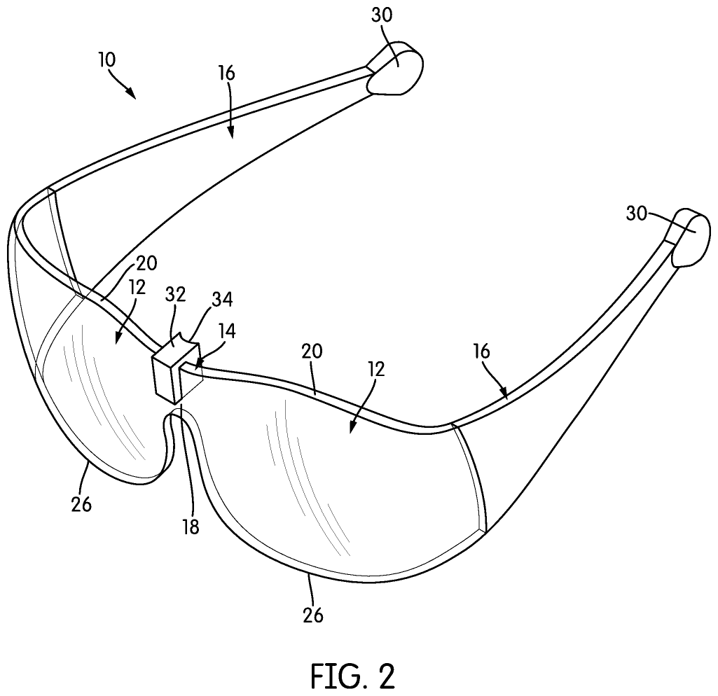

FIG. 1 is a perspective view of a patient in a reclining position wearing safety eyewear, generally indicated at 10, according to an embodiment of the present invention, and FIG. 2 is a perspective view of the safety eyewear 10 in isolation. In the following description of the safety eyewear 10, and of safety eyewear according to other embodiments of the invention, medical directional terms, such as “superior,” “inferior,” “medial,” “lateral,” “anterior,” and “posterior” will be used to refer to the positions of certain elements of the safety eyewear 10 relative to the body of the patient by whom they are worn. These terms should be construed to have their ordinary medical meanings.

Generally speaking, the safety eyewear 10 has many of the features of traditional eyeglasses: two lenses 12 held by a frame 14 that has a pair of temple arms 16, one arm 16 extending in the posterior direction from each side of the frame 14. The frame 14 includes a bridge 18 intended to fit over the bridge of the nose. However, as will be described in greater detail below, certain of these elements have been adapted to allow the safety eyewear 10 to remain in a stable position on the face when the patient is in a reclining or fully supine position.

With respect to the function and use of the safety eyewear 10, it should be understood that although some portions of this description may refer to the wearer of the safety eyewear 10 as a patient, and may describe certain hazards associated with dental treatment, the safety eyewear 10 may be worn by anyone needing eye protection in a reclining or supine position.

In the illustrated embodiment, the lenses 12 are integral and contiguous with the frame 14. For example, the lenses 12 may comprise thinned, at least translucent sections of the same material of which the frame 14 is made. Alternatively, the lenses 12 may be the same thickness as the frame 14. In this particular case, the frame 14 and the lenses 12 are both made of a plastic, such as polycarbonate, and are transparent to at least some wavelengths of light (although they may filter other wavelengths out). However, in other embodiments, the lenses 12 may be made of a different material than the frame 14 and may be placed in the frame 14 as is done with traditional eyewear. In general, the frame 14 and lenses 12 may be made of any suitable materials. In particular, the frame 14 may be made of metal, plastic, or another such suitable material. With respect to materials, it is helpful if the lenses 12, frame 14, and other components of the safety eyewear 10 can survive disinfection with standard chemical disinfectants without damage. While the particular disinfectants may vary, isopropanol in concentrations of 70% or greater is one particularly common disinfectant.

The lenses 12 may be tinted or have components to render them capable of polarizing light, filtering light (either generally or in particular wavelength ranges), or blocking certain wavelengths of light altogether. They may include specific coatings, like dichroic coatings, for the purpose of filtering the incoming light. For example, the lenses 12 may be pigmented or have coatings to filter the specific wavelengths or ranges of wavelengths of light used to cure composite resin, cements, and adhesives. As another example, the lenses 12 could be made to block specific wavelengths of laser light. More generally, the lenses 12 may be made with a significant tint so that the patient's view of the proceedings is significantly dimmed.

The lenses 12 and frame 14 may, in some cases, be made to comply with specific regional and local standards for safety eyewear, such as light filtering standards or impact resistance standards. However, they need not comply with any particular standard. In this description, the term “safety eyewear” refers to the general function of the eyewear, and does not necessarily imply compliance with any particular safety standard. Regardless of particular standards, it is helpful if the lenses 12 and frame 14 can sustain an impact of, e.g., at least about 1 kilogram of mass dropped from a height of about one meter without breaking.

As can be appreciated from the view of FIG. 1, the lenses 12 are relatively large, such that they cover the entire region around the eyes. Specifically, the superior edges 20 of the lenses 12 extend above the brow 24 and the inferior edges 26 of the lenses 12 extend below the orbits 28, to positions at or near the cheekbones. In general, the lenses 12 and frame 14 curve toward the face; laterally, the lenses 12 and frame 14 wrap around the head, all terminating in close proximity to the face, e.g., 1-2 mm from the face, although in some cases, the lenses 12 may rest on and make a near-seal around the face. The proximity of the edges of the lenses 12 to the face can help to prevent ingress of foreign material under the lenses 12.

As those of skill in the art will appreciate, in dental procedures, it may be more important that the inferior edges 26 of the lenses 12 and frame 14 are closely proximate to the face, because the inferior edges 26 are closer to the mouth. Thus, while curvature in the posterior direction, toward the face, may be provided in the superior edges 20, it may not be necessary in all embodiments.

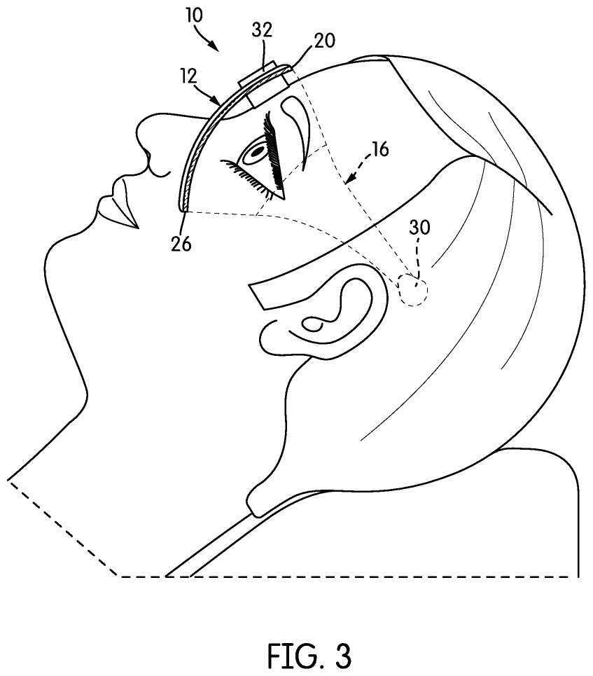

FIG. 3 is a partially cut-away side view of the safety eyewear 10, shown on a patient. As shown, the curvature of the lenses 12 places their optical centers a distance from the eye. As shown in FIG. 3, that distance is sufficient to accommodate long eyelashes. The curvature of the lenses 12 and the proximity of the inferior edges 26 to the face are also shown in FIG. 3. More particularly, in the view of FIG. 3, the inferior edges 26 of the lenses 12 rest against the face. For the reasons explained above, in this particular embodiment, the superior edges 20 do not come as close to the face, but in other embodiments, they may.

In some embodiments, the curvature of the lenses 12 could be more sharply parabolic than what is illustrated in FIG. 3. Additionally, although the frame 14 and lenses 12 have a single, continuous curvature in the illustration of FIG. 3, in some embodiments, the frame 14 and lenses 12 may have two or more distinct curvatures. For example, one or both of the frame 14 or the lenses 12 may curve more sharply in the posterior direction toward the inferior edge 20 than at the center or near the superior edge 26.

In order to promote comfort, the superior and inferior edges 20, 26 of the frame 14 may be covered or coated with thin layer of a foam or solid rubber in order to increase patient comfort at contact points with the face, if the lenses 12 contact the face.

In the illustrated embodiment, the lenses 12 of the safety eyewear 10 are integral with and meld into the frame 14. For that reason, the lenses 12 are described above as having certain curvature laterally, superiorly, and inferiorly. In other embodiments of the invention, particularly if the safety eyewear comprises lenses inset into a traditional frame, it may be the frame that has the curvature, the lenses, or a combination of the lenses and the frame. In other words, it is not critical which components have the curvature described above, so long as the eyewear as a whole comes close to or rests on the face in order to prevent ingress of debris and also provides enough space around the centers of the lenses 12 for the eyelashes.

As shown particularly in FIGS. 1 and 2, the frame 14 bends approximately 90° at its sides to form each of the temple arms 16. In the illustrated embodiment, there are no hinges between the frame 14 and the temple arms 16. Instead, to the extent that flexibility between these components is needed, the material of which the frame 14 and temple arms 16 is made is flexible enough to allow the temple arms 16 to deflect. Of course, in other embodiments, hinges may be provided between the frame 14 and the temple arms 16, but the absence of a hinge may simplify the manufacture of the safety eyewear 10 and simplify disinfection procedures once the safety eyewear 10 is in use.

As can be seen in FIGS. 1 and 3, the temple arms 16 are generally straight as they extend in the posterior direction, tapering in height from the height of the frame down to a narrower profile, and terminating in large, generally round temple pads 30 with no bend to hook over the ears.

The temple pads 30 may be made of a soft rubber, such as silicone rubber, both to provide comfort for the patient and, potentially, to add to the ability of the temple arms 16 to grip and stabilize the safety eyewear 10. Typically, the temple pads 30 bear against the scalp. The lack of a bend over the ears may reduce the chance that pressure on the temple arms 16 exerted by a headrest will push the eyewear 10 forward, away from the face.

The safety eyewear 10 includes another feature that stabilizes the frame 14 on the face in reclining and supine positions: a nasion pad 32. Located on the superior interior edge of the bridge 18 and extending in the posterior direction, toward the face, the nasion pad 32 is positioned to rest against the patient's nasion, and in so doing, to provide greater stability for the frame 14. As will be described below in more detail, the inward face 34 of the nasion pad 32 may have curvature appropriate for the nasion area.

Like the temple pads 30, the nasion pad 32 may be formed of a soft rubber, such as a silicone rubber, for contact with the face. In some embodiments, that soft rubber may be formed over a stiff or resilient metal or plastic reinforcement that helps the nasion pad 32 to retain its shape or enhances the ability of the nasion pad 32 to remain on the bridge 18.

There are a number of ways of attaching nasion pads to the frame 14. For example, if the frame 14 is made of a plastic or hard rubber, a nasion pad may be co-molded with the frame 14 itself. Nasion pads may also be attached after molding by various means, ranging from pressure-sensitive adhesive to cooperating engaging structures carried by the nasion pad and the frame 14. A nasion pad may be permanently attached to the frame 14, or it may be temporarily attached to the frame. Additional methods for attaching a nasion pad 32 will be described below in more detail.

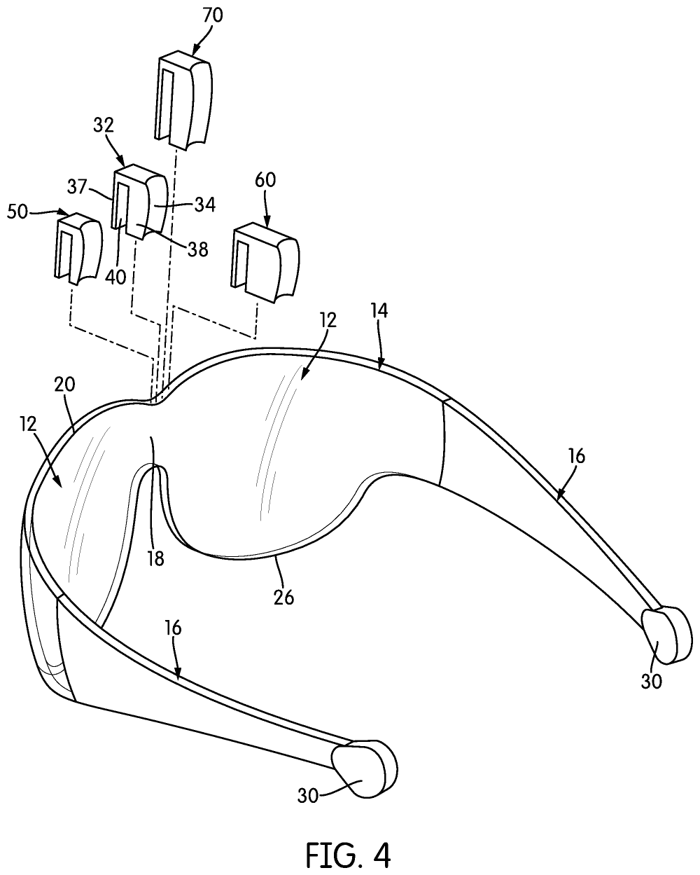

FIG. 4 is an exploded perspective view of the safety eyewear 10, showing the nasion pad 32 exploded away from the frame 14. As can best be seen in FIG. 4, in the safety eyewear 10, the nasion pad 32 has the form of an inverted U, with two depending legs 37, 38 separated by a gap 40. One of the legs 38 carries the inward face 34. Essentially, the nasion pad 32 clips onto the frame 14, inserting over the superior edge of the bridge 18. In some cases, friction between the bridge 18 and the nasion pad 32 may hold the nasion pad 32 in place. In other cases, a resilient reinforcing member within the nasion pad 32 may bias the two legs 37, 38 closer to one another so that the nasion pad 32 exerts force on the frame 18 to remain in place. In yet other embodiments, pressure-sensitive adhesive or other such means may be used. In some embodiments, markings may be provided on the bridge 18 indicating, either generally or specifically, the correct position for the nasion pad 32.

The inward face 34 of the nasion pad 32 has a complex curvature in order to at least generally match the curvature of the nasion area, as was noted briefly above. Specifically, the inward face 34 has a concave curvature in the lateral direction, and a convex curvature in the superior-inferior direction. Of course, it should be understood that the nasion pad 32 need not make contact with the entire nasion and its surrounding area; even point contact in this area may be sufficient to stabilize the safety eyewear 10.

As shown in FIG. 4, one advantage of a nasion pad 32 with these features is relatively easy interchangeability. Instead of disinfecting the nasion pad 32 and the area around it, a fresh nasion pad 32 may be provided for each patient, with used nasion pads 32 either being discarded or disinfected separately. Additionally, the interchangeability allows a clinician to select nasion pads with different sizes and characteristics for different patients. FIG. 4 illustrates four different nasion pads, including the nasion pad 32 of the previous figures, all of them adapted to be used with the safety eyewear 10. More specifically, FIG. 4 illustrates a thinner nasion pad 50, a thicker nasion pad 60, and a longer nasion pad 70 that extends farther down along the bridge 18. The overall shape of the nasion pad 32 and its curvatures, especially the curvatures of the inward face 34, may also be modified.

In safety eyewear 10, a combination of features of the safety eyewear 10 provides stability on the face, including the curvature of the lenses 12, the curvature of the frame 14, the shape and extent of the temple arms, and the nasion pad 32. However, using some of those features alone may be sufficient to improve stability on the face. In particular, a nasion pad 32 may provide additional stability in reclining and supine positions when used alone.

In some embodiments of the invention, a stand-alone nasion pad 32, 50, 60, 70 may be sold and retrofit to eyewear that does not have all of the features of the safety eyewear 10 described here. In those cases, the size of the gap 40 and extent of the two depending legs 37, 38 may be modified to fit different kinds of eyewear. Alternatively, the material of the legs 37, 38 may be such that the nasion pad 32 can be stretched over the bridge of the frame to fit a variety of different types of eyewear. The presence of the nasion pad 32 may provide some additional stability in reclining and supine positions.

As is typical for eyewear, the safety eyewear 10 may be made in various sizes and dimensions in order to accommodate different size faces, including both pediatric and adult sizes. Where descriptors such as “above the brow” and “at the cheekbone” are used in this description to indicate the relative extent of the safety eyewear 10 on the face, those of ordinary skill in the art may use average anthropometric data for, e.g., a 50^{th} percentile male, a 50^{th} percentile female, an 80^{th} percentile male, an 80^{th} percentile female, etc., in order to establish the absolute sizes and dimensions.

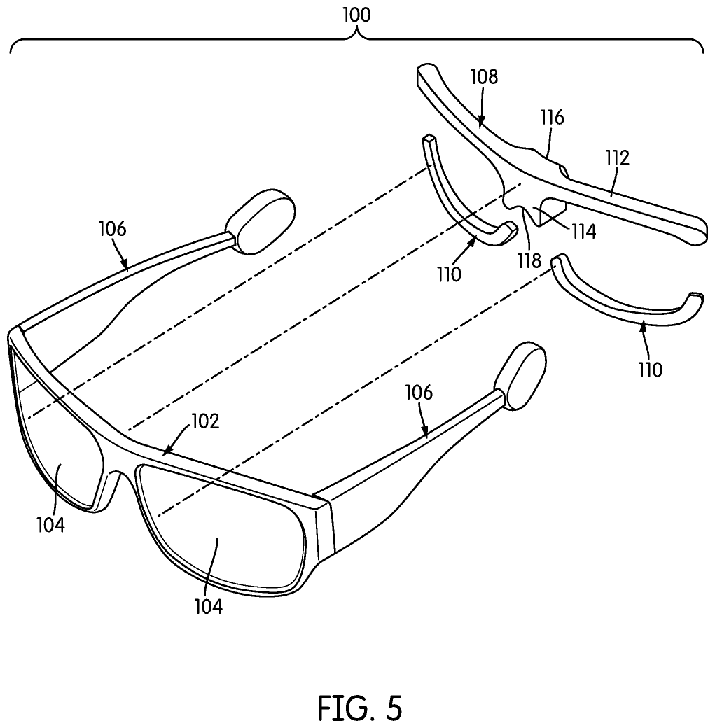

FIG. 5 is an exploded perspective view of safety eyewear, generally indicated at 100, according to another embodiment of the invention. The safety eyewear 10 has a frame 102 of more traditional shape with inset lenses 104. Temple arms 106 are connected to the lateral aspects of the frame 102. The temple arms 106 are generally similar to the temple arms 16 described above. The lenses 104 may be transparent, or they may be made to filter various wavelengths of light, as described above.

The frame 102 is not a wrap-around frame and is somewhat more planar in its extent. Notably, the frame and lenses 102, 104 do not have superior-inferior curvature that causes them to terminate close to the face. Therefore, the safety eyewear 100 would typically stand-off some distance from the face.

Thus, as shown in FIG. 5, the safety eyewear 100 includes a superior frame support 108 and a pair of crescent-shaped inferior frame supports 110. The frame supports 108, 110 that are attached to the patient-facing side of the frame 102 and are intended to rest against and to support the frame 102 against the patient's face.

The upper, lateral portions 112 of the superior frame support 108 follow the curvature of the upper member of the frame. The superior frame support 108 also includes a depending central portion 114 that is thicker than the upper, lateral portions 112. The central portion 114 includes an inwardly-facing surface 116 that is intended to rest against the nasion and act as a nasion pad. The inwardly-facing surface 116 may have the kind of complex curvature described above with respect to the nasion pad 32. A lower surface 118 of the central portion 114 is contoured for the bridge of the nose. This lower contour could be provided on the nasion pads 32, 50, 60, 70 described above in some cases.

The pair of crescent-shaped inferior frame supports 110 attaches to the lower portion of the frame 102, around the bottoms of the lenses 104. Together, the frame supports 108, 110 provide positive contact with the face and more stability for the eyewear 100 in reclining and supine positions. Additionally, the frame supports 108, 110 prevent ingress of foreign material into the gaps between the face and the frame 102.

The frame supports 108, 110 could be co-molded with the frame 102, if the frame is plastic, or attached to it permanently or temporarily after manufacture of the frame 102. A pressure-sensitive adhesive or a traditional one- or two-part adhesive could be used to secure the components together. In some cases, the frame supports 108, 110 could include clip structures to clip over the frame 102, much as the nasion pad 32 does with the safety eyewear 10 described above. The materials of which the frame 102, lenses 104, temple arms 106 and frame supports 108, 110 are made are not critical, so long as the safety eyewear 100 provides appropriate protection for the patient, and so long as the components can survive disinfection with typical chemical disinfecting agents.

Like the nasion pad 32 described above, the superior and inferior frame supports 108, 110 could be provided as a kit for retrofitting a more traditional type of eyewear with features useful for stability in reclining and supine positions.

Of course, features from the various embodiments of safety eyewear 10, 100 are not necessarily mutually exclusive. For example, another embodiment of safety eyewear according to an embodiment of the invention could have both a nasion pad 32 as described above with respect to the safety eyewear 10 and inferior frame supports 110 as described above with respect to the safety eyewear 100.

While the invention has been described with respect to certain embodiments, the description is intended to be exemplary, rather than limiting. Modifications and changes may be made within the scope of the invention, which is defined by the appended claims.

What is claimed is:

1. Safety eyewear, comprising:
a frame configured to rest over a face, the frame including a pair of lenses separated from one another by a bridge, one of both of the frame and the pair of lenses curving in a posterior direction along at least inferior edges thereof;
left and right temple arms extending generally straight in the posterior direction from respective left and right sides of the frame; and
a nasion pad coupled to the bridge, the nasion pad extending in the posterior direction from the bridge.

2. The safety eyewear of claim 1, wherein one or both of the frame and the pair of lenses are curved laterally.

3. The safety eyewear of claim 2, wherein the left and right temple arms each terminate in a temple pad.

4. The safety eyewear of claim 3, wherein the temple pads are rounded.

5. The safety eyewear of claim 2, wherein the nasion pad comprises:
a clip having an outward depending leg and an inward depending leg separated by a gap, the inward leg having a depth greater than a depth of the outward leg; and
an inward face on the inward leg, the inward face having a concave curvature in a lateral direction and a convex curvature in a superior-inferior direction.

6. The safety eyewear of claim 2, wherein the lenses are integral and contiguous with the frame.

7. The safety eyewear of claim 2, wherein, when worn, a superior edge of the eyewear lies above the brow of the face and an inferior edge of the eyewear lies at the cheekbone of the face.

8. The safety eyewear of claim 2, wherein one of both of the frame and the pair of lenses curve in a posterior direction along superior edges thereof.

9. The safety eyewear of claim 1, wherein the nasion pad has an inward face that is concave in a lateral direction and convex in a superior-inferior direction.

10. A nasion pad for eyewear, comprising:
a clip having an outward depending leg and an inward depending leg separated by a gap, the inward leg having a depth greater than a depth of the outward leg; and
an inward face on the inward leg, the inward face having a concave curvature in a lateral direction and a convex curvature in a superior-inferior direction.

11. The nasion pad of claim 10, wherein the outward leg and the inward leg are resiliently biased into positions closer to one another.

12. The nasion pad of claim 10, wherein the nasion pad is formed of a foam or rubber.

13. Safety eyewear, comprising:
a frame configured to rest over a face, the frame including a pair of lenses separated from one another by a bridge, one of both of the frame and the pair of lenses curving in a posterior direction along at least inferior edges thereof, and one or both of the frame and the pair of lenses are curving laterally around the face;
left and right temple arms extending generally straight in the posterior direction from respective left and right sides of the frame; and
a nasion pad coupled to the bridge, the nasion pad extending in the posterior direction from the bridge and including:
a clip having an outward depending leg and an inward depending leg separated by a gap, the inward leg having a depth greater than a depth of the outward leg, and
an inward face on the inward leg, the inward face having a concave curvature in a lateral direction and a convex curvature in a superior-inferior direction.

14. The safety eyewear of claim 13, wherein the outward leg and the inward leg of the nasion pad are resiliently biased into positions closer to one another.

15. The safety eyewear of claim 13, wherein the lenses are integral and contiguous with the frame.

16. The safety eyewear of claim 13, wherein, when worn, a superior edge of the eyewear lies above the brow of the face and an inferior edge of the eyewear lies at the cheekbone of the face.
